(19) United States
(12) Patent Application Publication
(10) Pub. No.: US 2017/0088815 A1
(43) Pub. Date: Mar. 30, 2017

(54) CONTROLLABLE FORMATION OF MICROVASCULAR NETWORKS USING SACRIFICIAL MICROFIBER TEMPLATES

(71) Applicant: THE TRUSTEES OF THE STEVENS INSTITUTE OF TECHNOLOGY, Hoboken, NJ (US)

(72) Inventors: Hongjun Wang, Millburn, NJ (US); Chao Jia, Hoboken, NJ (US)

(73) Assignee: THE TRUSTEES OF THE STEVENS INSTITUTE OF TECHNOLOGY, HOBOKEN, NJ (US)

(21) Appl. No.: 15/086,896

(22) Filed: Mar. 31, 2016

Related U.S. Application Data
(60) Provisional application No. 62/140,840, filed on Mar. 31, 2015.

(51) Int. Cl. C12N 5/00

(52) U.S. Cl. CPC C12N 5/0075

(57) ABSTRACT

The present disclosure relates to fabricating sacrificial microfiber templates from any biocompatible and resorbable materials depending on the time needed for dissolving the microfiber template to free the endothelial tube with open lumen. Microfiber networks with distinct patterns and defined diameters initially serve as a template to support the growth of vascular cells (endothelial cells or their progenitor cells, or combined with mural cells such as pericytes) and then dissolve to form an empty endothelium lumen. The incorporation of sacrificial microfiber networks encapsulated with vascular cells into 3D cell-rich constructs allows for the creation of various vascularized tissues.

### CROSS-REFEREMCE TO RELATED APPLICATION

The present application claims priority to U.S. Provisional Patent Application Ser. No. 62/140,840, filed Mar. 31, 2015, the disclosure of which is incorporated by reference herein in its entirety.

### FIELD OF THE INVENTION

The exemplary embodiments relate generally to biomedical engineering, and, more specifically, to tissue engineering.

### BACKGROUND OF THE INVENTION

Despite advances in creation of avascular cartilage and thin skin tissues, it remains highly challenging to create tissues and organs with large volume and a hierarchical structure using the tissue-engineering approach. Among various challenges in regulating tissue formation, the introduction of a functional microvascular network into tissue constructs has been recognized as a key step; most cells in the natural tissues need to remain within about 200 μm of the nearest capillary for effective and sufficient transport of nutrients, exchange of oxygen, and removal of metabolic wastes. In addition to the need for a supply of nutrition, for implanted tissue constructs to function properly, efficient vascularization is crucial for such implanted tissue constructs to transmit and receive systemic factors and hormones in order to communicate with the whole organism.

### SUMMARY OF THE INVENTION

In view of the foregoing background, a method of fabricating a vascular structure is disclosed. The method includes providing a microfiber template comprising a plurality of interconnected microfibers configured to support adhesion and spreading of endothelial cells, the plurality of interconnected microfibers being made from at least one biocompatible and biodegradable material, and placing the microfiber template in a culture that includes endothelial cells and/or endothelial progenitor cells. The endothelial cells and/or endothelial progenitor cells seed onto the microfiber template to form at least one endothelial layer over the microfiber template, and the microfiber template partially or fully degrades after a set period of time, leaving a vascular structure from the at least one endothelial layer.

The exemplary embodiments relate to an approach for forming a functional microvascular network that can be easily integrated into tissue-engineered tissue constructs. In the exemplary embodiments, endothelial cells and endothelial progenitor cells isolated from vessels or derived from stem cells are seeded and cultured on the surface of a sacrificial microfiber network template (e.g., 5-100 μm in diameter) until they grow into a confluent layer and, subsequently, form a microvascular structure with open lumen and well controlled pattern upon dissolution of the fiber template. Endothelial cells are capable of forming the intercellular junctions that assure the structural integrity of endothelial tubes after the removal of microfiber template. The microfiber template may be made from any biocompatible and biodegradable materials and may have any desired shapes or patterns. In an embodiment, the diameters and organization patterns may emulate natural microvessels (e.g., capillaries, arterioles and venules) by introducing vascular supporting mural cells such as pericytes over the outside of the endothelial layer. Selection of materials for microfiber template may be based on a required degradation time window (e.g., 2-3 weeks) in addition to their supportiveness to the adhesion and proliferation of endothelial cells or endothelial progenitor cells. The exemplary embodiments offer control of the microvascular size and the network pattern by the template fibers, keep the microvessel open (e.g., the exemplary embodiments demonstrate good patency), and have a high potential to be integrated into three-dimensional (“3D”) tissue-engineered constructs.

### BRIEF DESCRIPTION OF FIGURES

The patent or application file contains at least one drawing executed in color. Copies of this patent or patent application publication with color drawing(s) will be provided by the Office upon request and payment of the necessary fee.

For a more complete understanding of the present invention, reference is made to the following detailed description of an embodiment considered in conjunction with the accompanying drawings, in which:

FIG. 1 is a diagram illustrating template-guided formation of microvessels in accordance with an exemplary embodiment of the present invention;

FIG. 2A is a diagram illustrating an exemplary microetching printing (“μEP”) system;

FIG. 2B is a 100 μm-scale stereomicroscopic image of microfiber that may be made using the system shown in FIG. 2A;

FIG. 2C is a 100 μm-scale image of Phalloidin staining showing mouse endothelial cells (MS-1 cells) covering the surface of microfibers that may be made using the system shown in FIG. 2A;

FIG. 2D is a 100 μm-scale image of 4′,6-diamidino-2-phenylindole (DAPI) staining showing MS-1 cells covering the surface of microfibers that may be made using the system shown in FIG. 2A;

FIG. 2E is a 100 μm-scale merged image of Phalloidin and DAPI staining showing MS-1 cells covering the surface of microfibers that may be made using the system shown in FIG. 2A;

FIG. 2F is a 100 μm-scale image of CD31 antibody staining showing MS1-cells forming confluent cell layer and covering the surface of microfibers that may be made using the system shown in FIG. 2A;

FIG. 2G is a 100 μm-scale image of DAPI staining showing MS1-cells forming confluent cell layer and covering the surface of microfibers that may be made using the system shown in FIG. 2A;

FIG. 2H is a 100 μm-scale merged image of CD31 antibody and DAPI staining showing MS1-cells forming confluent cell layer and covering the surface of microfibers that may be made using the system shown in FIG. 2A;

FIG. 21 is a first 20 μm-scale confocal image of CD31 antibody and DAPI staining showing lumen formation after polymer degradation according to the system shown in FIG. 2A;

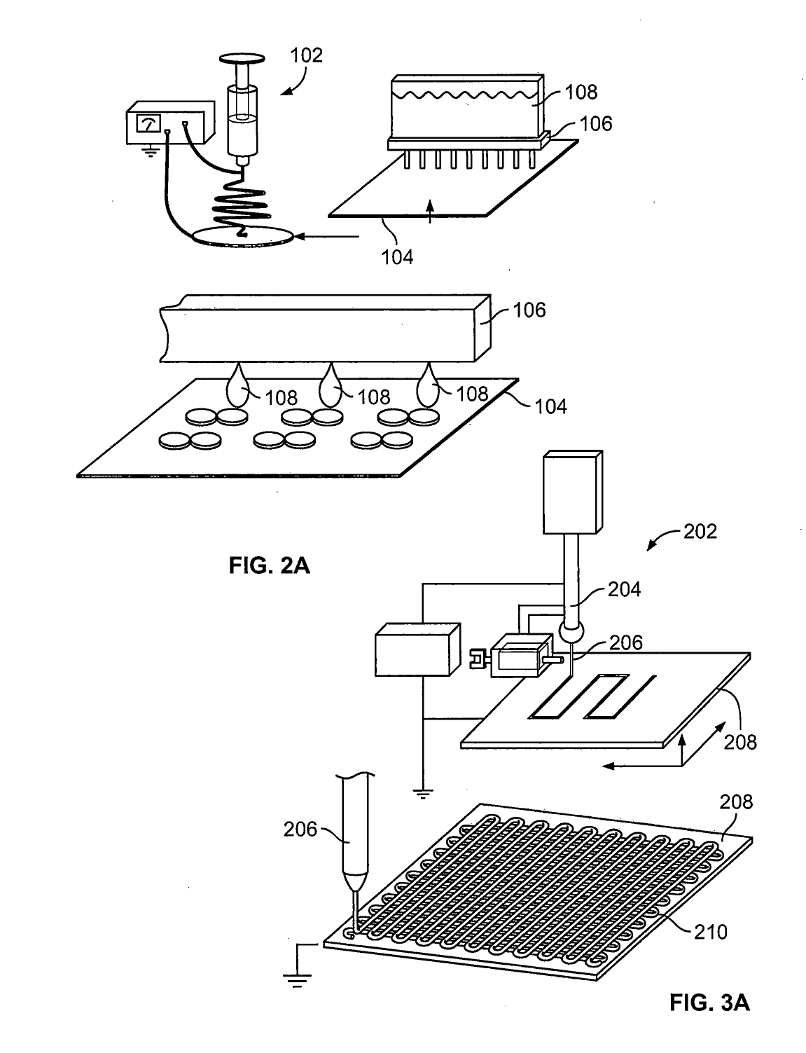

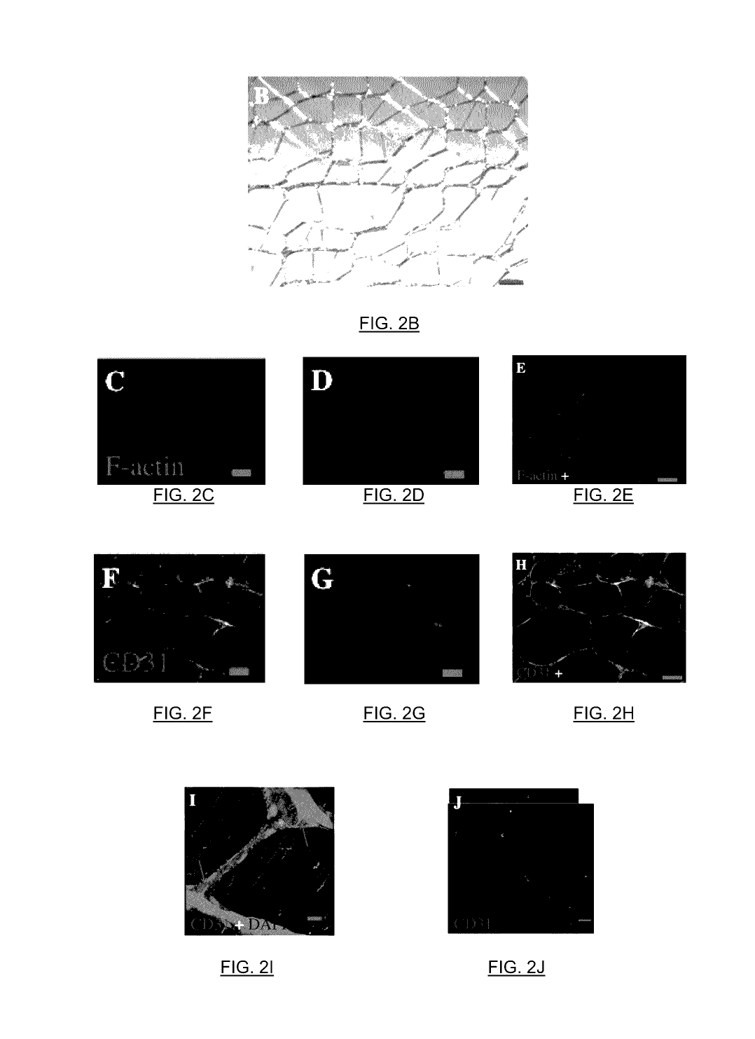

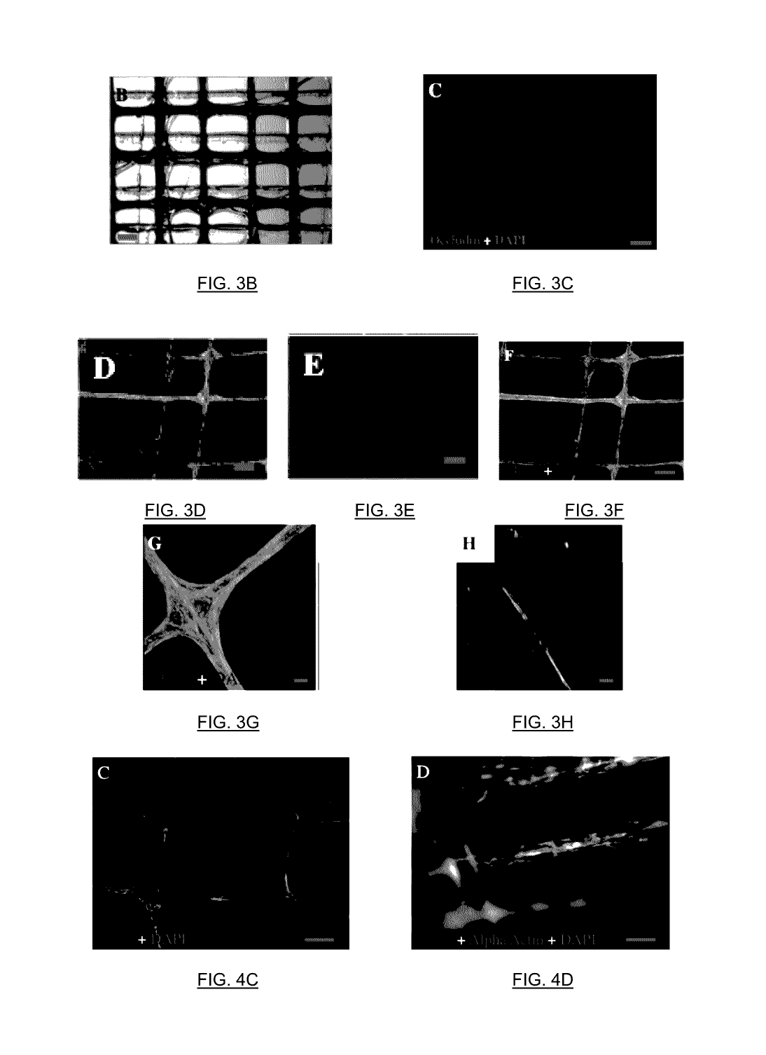

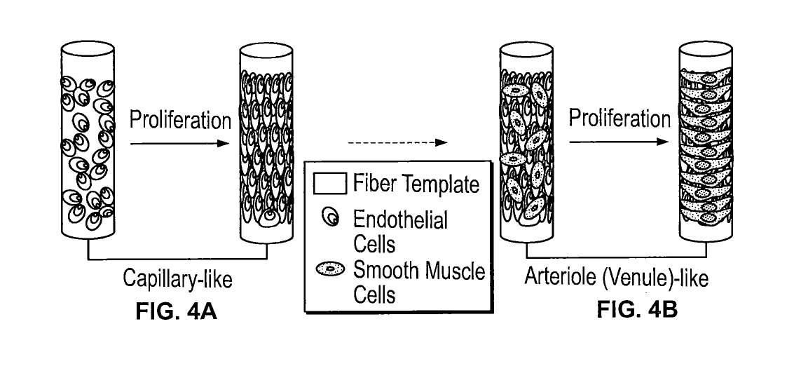

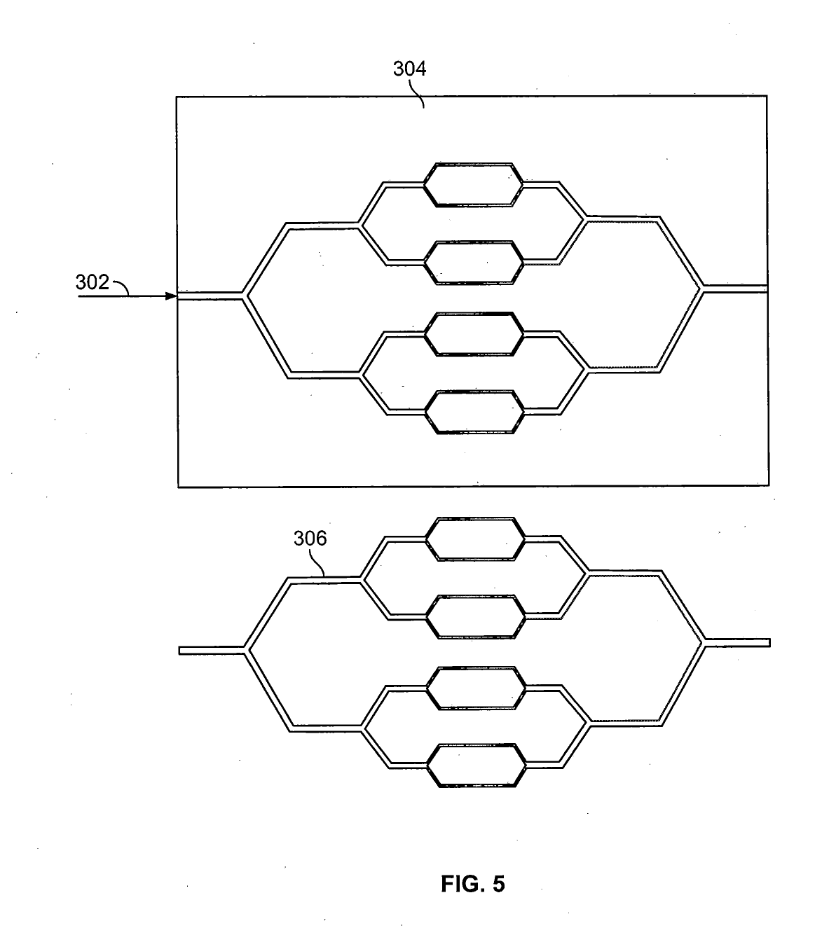

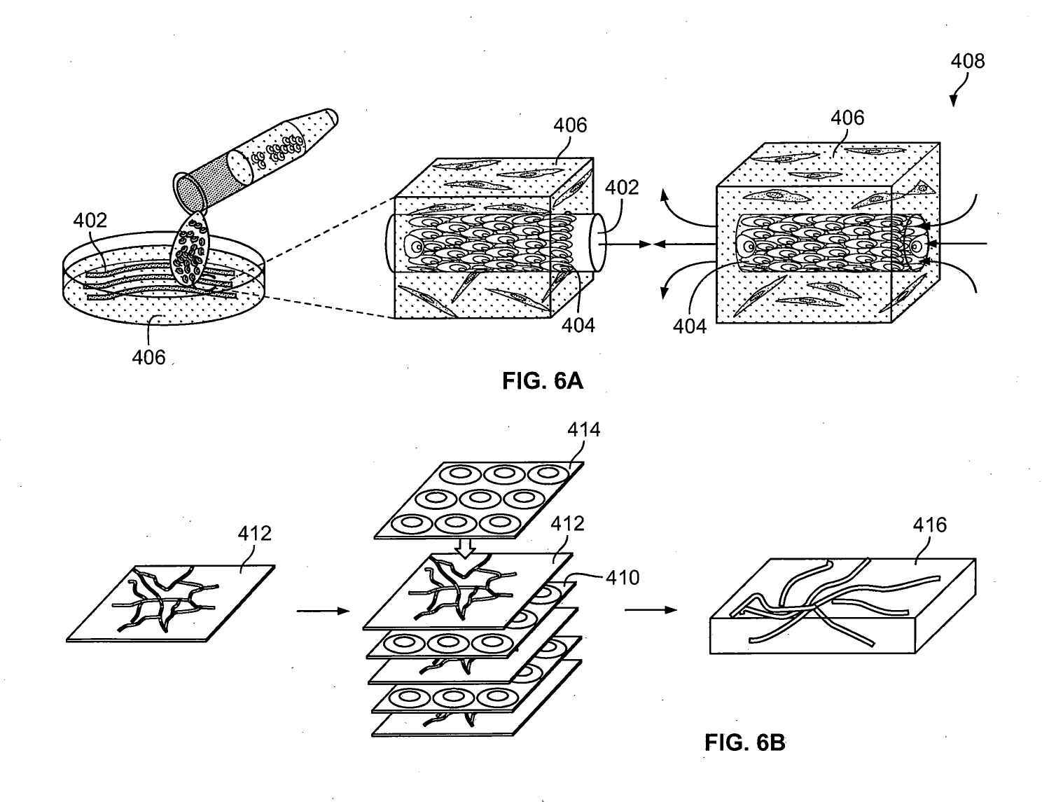

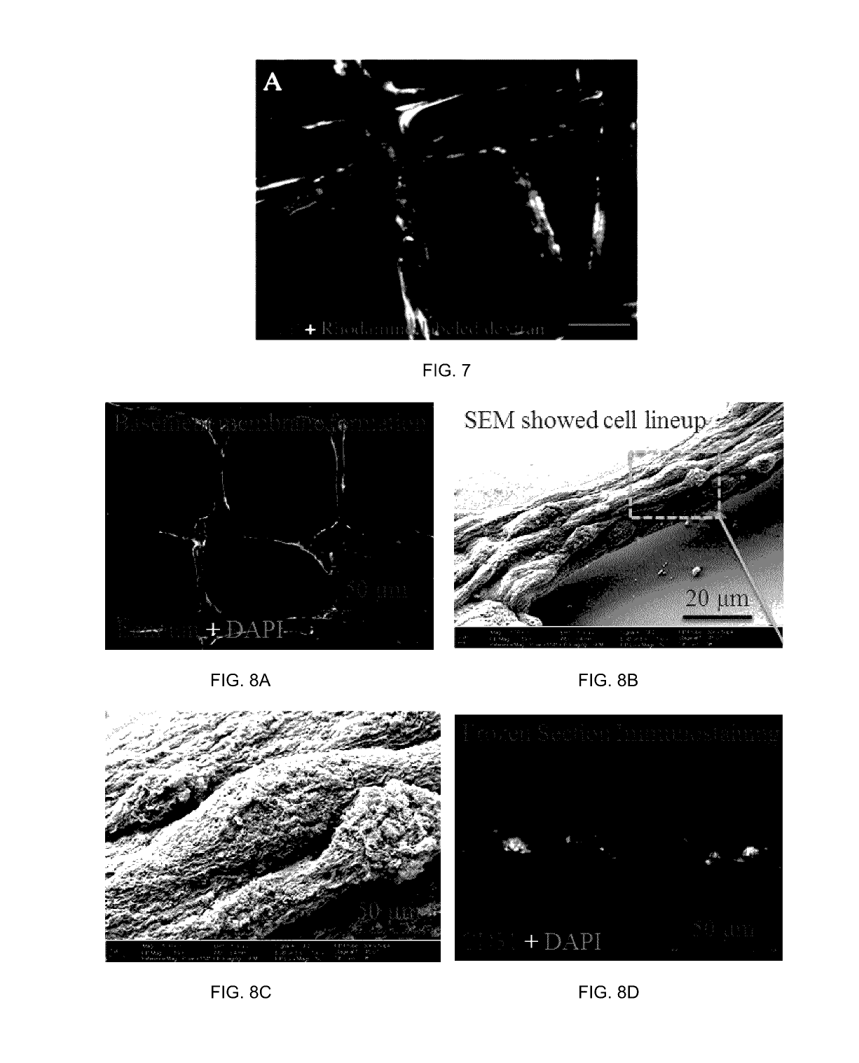

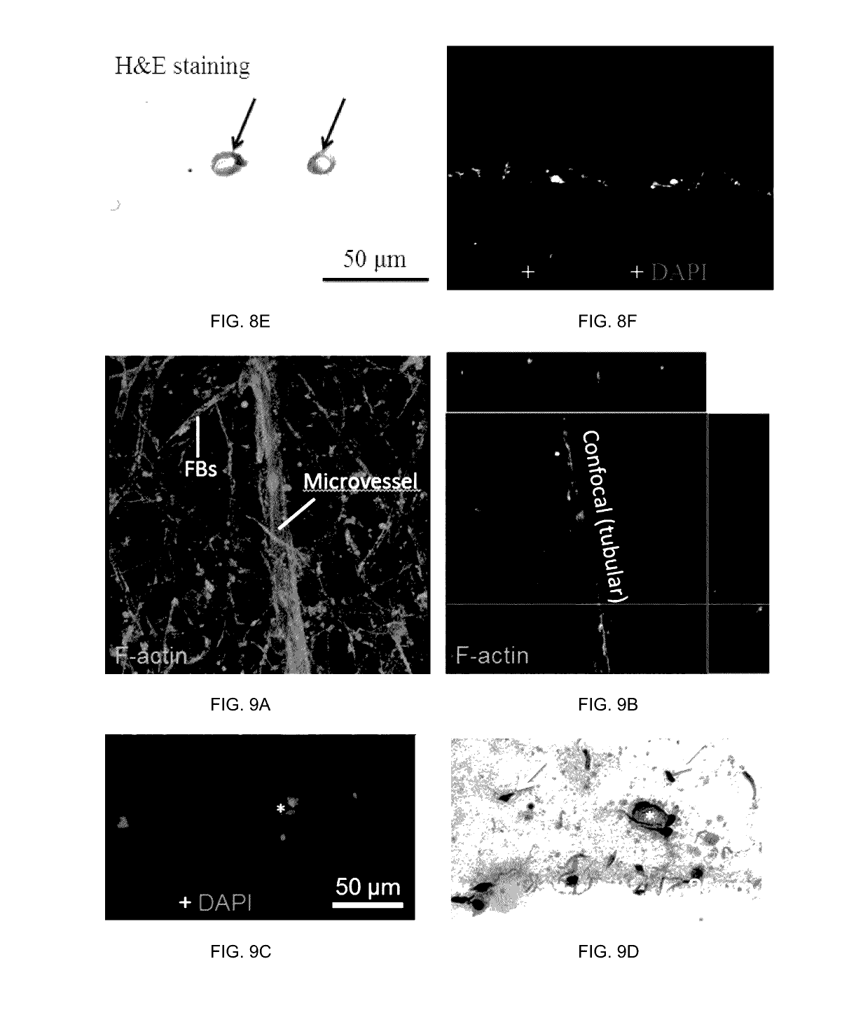

FIG. 2J is a second 20 μm-scale confocal image of CD31 antibody and DAPI staining showing lumen formation after polymer degradation according to the system shown in FIG. 2A;

FIG. 3A is a diagram illustrating an exemplary electrostatic deposition prototyping (“EDP”) system;

FIG. 3B is a 100 μm-scale stereomicroscopic image of microfiber that may be made using the system shown in FIG. 3A;

FIG. 3C is a 50 μm-scale merged image showing Occludin antibody and DAPI staining showing tight junctions formed by MS-1 cells made using the system shown in FIG. 3A;

FIG. 3D is a 100 μm-scale image of CD31 antibody staining showing MS1-cells forming confluent cell layer and covering the surface of microfibers that may be made using the system shown in FIG. 3A;

FIG. 3E is a 100 μm-scale image of DAPI staining showing MS1-cells forming confluent cell layer and covering the surface of microfibers that may be made using the system shown in FIG. 3A;

FIG. 3F is a 100 μm-scale merged image of CD31 antibody and DAPI staining showing MS1-cells forming confluent cell layer and covering the surface of microfibers that may be made using the system shown in FIG. 3A;

FIG. 3G is a first 20 μm-scale confocal image of CD31 antibody and DAPI staining showing lumen formation after polymer degradation according to the system shown in FIG. 3A;

FIG. 3H is a second 20 μm-scale confocal image of CD31 antibody and DAPI staining showing lumen formation after polymer degradation according to the system shown in FIG. 3A;

FIG. 4A is a diagram illustrating template-guided formation of capillary-like tissues according to an exemplary embodiment of the present invention;

FIG. 4B is a diagram illustrating template-guided formation of arteriole-like or venule-like tissues according to an exemplary embodiment of the present invention;

FIG. 4C is a 100 μm-scale merged image of CD31 antibody and DAPI staining showing intracellular junction formation between cells according to an exemplary embodiment;

FIG. 4D is a 50 μm-scale merged image of a-smooth muscle actin antibody and CD31 antibody and DAPI staining showing mouse smooth muscle cells attached to an endothelium according to an exemplary embodiment;

FIG. 5 schematically illustrates creation of a patterned microfiber template using an exemplary microfluidic molding approach;

FIG. 6A is a diagram showing integrating an endothelial cell-coated microfiber template in a three-dimensional construct in accordance with a first embodiment of the present invention;

FIG. 6B is a diagram showing integrating an endothelial cell-coated microfiber template in a three-dimensional construct in accordance with a second embodiment of the present invention;

FIG. 7 is a 50 μm-scale image of a top view of infiltration of Rhodamine-labeled dextran through an engineered vascular network;

FIG. 8A is a 50 μm-scale merged image of laminin-111 antibody and DAPI staining showing a basement membrane formation on a microfiber template in accordance with an embodiment of the present invention;

FIG. 8B is a 20 μm-scale image of a scanning electron microscopy (SEM) image of endothelial cells line up on microfiber template surface; ;

FIG. 8C is a 50 μm-scale enlarged view of a portion of the engineered vascular network shown in FIG. 8B;

FIG. 8D is a 50 μm-scale image of CD31 antibody and DAPI staining showing a harvested sample of a cultured microvascular network made in accordance with an embodiment of the present invention, the sample being frozen, sectioned and subject to immunostaining;

FIG. 8E is a 50 μm-scale image of the harvested sample shown in FIG. 8D, the sample being fixed, embedded, sectioned and subject to Hematoxylin and eosin staining, red arrows indicating the formed blood vessels;

FIG. 8F is a 50 μm-scale image of the harvested sample shown in FIG. 8D, the sample being frozen, sectioned and subject to immunostaining;

FIG. 9A is a first image of Phalloidin staining showing a co-culture of skin fibroblasts (FBs) and MS-1 encapsulated microfibers in collagen gel in accordance with the embodiments shown in FIGS. 8A and 8B;

FIG. 9B is a second image of Phalloidin staining showing a co-culture of skin fibroblasts (FBs) and MS-1 encapsulated microfibers in collagen gel in accordance with the embodiments shown in FIGS. 8A and 8B;

FIG. 9C is a 50 μm-scale image of CD31 antibody and DAPI staining showing the frozen sections of the co-culture shown in FIGS. 9A and 9B that has been subject to immunostaining; and

FIG. 9D is a 25 μm-scale image showing the cross-section of the co-culture shown in FIGS. 9A and 9B that has been subject to Hematoxylin and eosin staining, white arrows indicating the formed blood vessels.

### DETAILED DESCRIPTION OF THE INVENTION

The following disclosure is presented to provide an illustration of the general principles of the present invention and is not meant to limit, in any way, the inventive concepts contained herein. Moreover, the particular features described in this section can be used in combination with the other described features in each of the multitude of possible permutations and combinations contained herein.

All terms defined herein should be afforded their broadest possible interpretation, including any implied meanings as dictated by a reading of the specification as well as any words that a person having skill in the art and/or a dictionary, treatise, or similar authority would assign thereto.

Further, it should be noted that, as recited herein, the singular forms ‘a,’ “an,” and “the” include the plural referents unless otherwise stated. Additionally, the terms “comprises” and “comprising” when used herein specify that certain features are present in that embodiment, however, this phrase should not be interpreted to preclude the presence or additional of additional steps, operations, features, components, and/or groups thereof.

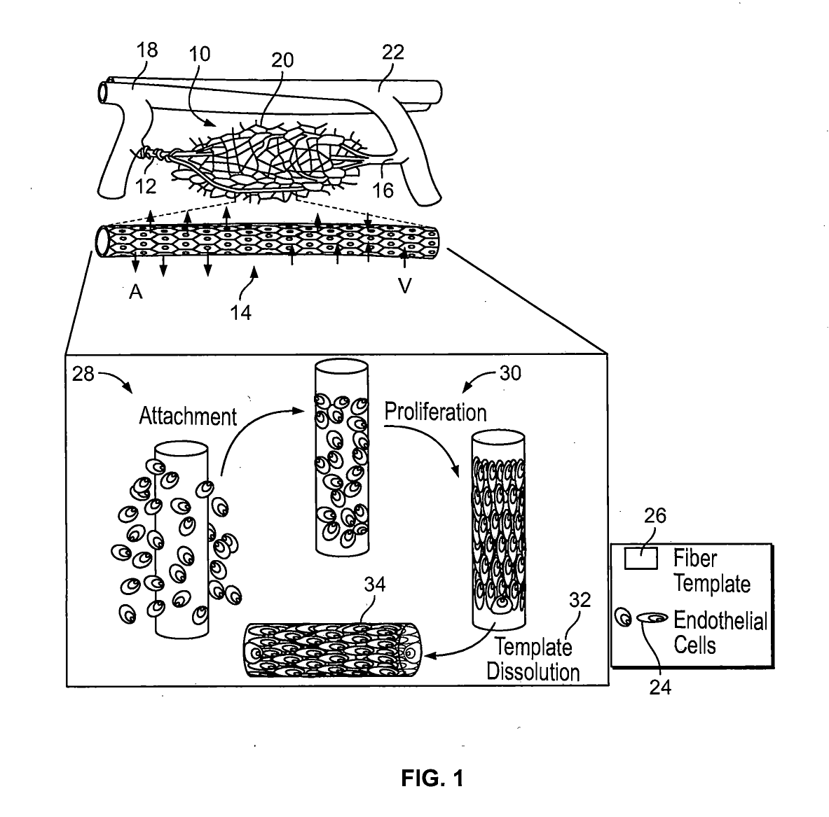

With reference to FIG. 1, in vivo microvascular networks 10 are mainly composed of arterioles 12, capillaries 14, and venules 16, in which arterioles 12 guide the bioactive molecule-enriched blood from artery 18 into the capillary bed 20, and venules 16 bring the blood with metabolic byproducts from the capillary bed 20 back to the main stream through the vein 22. Capillaries 14, composed of a single layer of endothelial cells 24 with diameters of 5-10 μm, are primarily responsible for nutrient and oxygen exchange through their permeable thin walls. FIG. 1 illustrates this flow, where fluid from an arteriole 14 (i.e., bioactive molecule-enriched blood) is squeezed out of the capillary 14 by blood pressure at A, and fluid (i.e., blood with metabolic byproducts from the capillary bed 20) reenters the capillary 14 at V via osmotic attraction to be carried out through a venule 16.

Arterioles 12 and venules 16 differ from capillaries 14 in that both have muscular walls. The diameter of arterioles 12 can be as small as 30 μm; the diameter of venules 16 can be as small as 8 μm.

The exemplary embodiments of the present disclosure relate to the formation of such microvessel structures through the use of microfiber templates 26 with an optimal surface for endothelial cells or progenitor cells to grow and an appropriate degradation rate to free the microvessels. As shown in FIG. 1, a microfiber template 26 is placed in a culture of endothelial cells 24, thereby allowing the endothelial cells 24 to attach to the microfiber template 26 (step 28). The endothelial cells 24 are then allowed to proliferate around the microfiber template 26 to create a layer of endothelial cells 24 that envelops the microfiber template 24 (step 30). Thereafter, the microfiber template 24 is allowed to dissolve (step 32), leaving a microvascular structure 34 composed of the layer of endothelial cells.

Microfiber templates may be fabricated using different techniques, such as through the use of microetching printing (“μEP”), as illustrated in FIG. 2A. In μEP, an electrospiner 102 fabricates a microfibrous matrix or mesh onto a medium 104. Thereafter, a printing head 106 loaded with etching solvent 108 prints the etching solvent 108 onto the medium 104 according to a predetermined pattern (i.e., the desired shape of the microfiber template). The etching solvent 108 then dissolves, either partially or completely, the portions of the microfibrous mesh to create a microfiber template according to the shape determined by the printing pattern.

Microetching printing is more thoroughly described in U.S. patent application Ser. No. 14/545,569 (Publication No. 2015/037489), the disclosure of which is incorporated by reference herein in its entirety.

Another technique for fabricating the microfiber template includes electrostatic deposition prototyping (“EDP”), as illustrated in FIG. 3A. As seen in FIG. 3A, an electrodeposition/electrospinning apparatus 202, which includes a syringe 204 fitted with a needle 206 positioned over an electrically grounded collector plate 208, is loaded with the polymer solution used to form the microfibers of the microfiber template. A voltage is applied to the needle 206 to allow it to spin the microfibers, and the apparatus 202 manipulates the syringe 204 to fabricate a patterned layer 210 of microfibers. Electrostatic deposition prototyping is more thoroughly described in U.S. patent application Ser. No. 14/157,602 (Publication No. 2014/0207248), the disclosure of which is incorporated by reference herein in its entirety.

Another technique includes microfluidic molding, as illustrated in FIG. 5, where polymer solution 302 is injected into a microfluidic mold 304 and allowed to set, thereby creating a microfiber template 306 whose shape conforms to that of the mold 304. It will be apparent to those of skill in the art that other techniques for the formation of microfiber templates may be possible without departing from the broader principles described herein.

Each of the above-described techniques may enable fabrication of various microfibers and microfiber networks from different biocompatible and resorbable materials (e.g., synthetic materials, natural materials, blended materials including both synthetic and natural elements) in a controlled manner. Microfluidic molding may also allow for readily creating hierarchical microfiber networks with multiple diameters, as shown in FIG. 5, similar to the dimensions of arterioles, capillaries and venules. μEP-microfiber may be used for formation of capillary networks, as illustrated in FIG. 2B, and EDP-microfiber and microfluidic molded microfiber networks may be used to form arterioles and venules, as illustrated in FIG. 3B. The microfibers themselves can be solid fiber, porous fiber, core-shell fiber, or other types of fiber depending upon the type of vascular network desired.

In order to support endothelial cells or endothelial progenitor cells to form a tubular network, the exemplary embodiments may use biocompatible and biodegradable polymers. The microfiber template should support the adhesion and spreading of endothelial cells and maintain their structural integrity prior to the formation of endothelial tube. Theoretically, any biocompatible and biodegradable materials may be used, including, but not limited to, synthetic polymers such as PCL, poly (L-lactic acid), poly (DL-lactic acid), poly (glycolic acid), poly(lactic-co-glycolic acid) (with different lactic acid-glycolic acid ratios: 95:5, 90:10, 85:15, 75:25, 50:50), poly(lactic-co-caprolactone) (with different lactic acid-caprolactone ratios: 90:10, 75:25, 50:50), poly (dioxanone), poly (esteramide), co-poly (oxalates), poly (carbonates), poly (glutamic-co-leucine), poly (ethylene), poly(ethylene glycol)-terephthalate/poly(butylene terephthalate), and poly (N-isopropylacrylamide), natural polymers such as collagen, gelatin, alginate, chitosan, fibrinogen, elastin, silk, polysaccharide, proteoglycans, hyaluronan, laminin, and fibronectin, or natural/synthetic blends. In one embodiment, a blend of polycaprolactone (PCL), collagen and poly(lactic-co-glycolic acid) (PLGA) may be used. PCL was used for its mechanical strength to support the cell attachment. Collagen may be used for its biological benefit for the cells to grow on. PLGA may be used for its tunable degradation based on the ratio between lactic acid and glycolic acid. Microfiber templates fabricated from both methods (e.g., μEP and EDP) may support the attachment of endothelial cells and have stimulatory effects on the proliferation and formation of intercellular junctions between endothelial cells, as illustrated in FIGS. 2B-J and 3B-H.

Both arterioles 12 and venules 16 differ from capillaries 14 in that arterioles 12 and venules 16 contain smooth muscle layers outside the luminal endothelium to provide unique mechanical and actuation function, as illustrated in FIGS. 4A and 4B. Based on the diameter of an arteriole or venule being in the range of 30 μm or larger, microfibers prepared from EDP and microfluidic-enabled microfibers may be used. The exemplary embodiments may be evaluated through the preparation of templates of polymeric microfiber networks using μEP, which may then have mouse endothelial cells (MS-1) cultured thereon. After 7 days, the culture may be fluorescently stained with TRITC-conjugated-Phalloidin for cytoskeletal stress fiber. The culture may also be stained with F-actin, as illustrated in FIGS. 2C, 2D, and 2E, or with and PECAM-1 (CD31), as illustrated in FIGS. 2F, 2G, 2H and 4C, to show the intercellular junctions. Based on staining, it may be observed that MS-1 not only attached to the exemplary microfiber surface, but also wrapped around the fiber to form a tubular structure with the cells tightly connected.

In the exemplary embodiments, MS-1 cells may be cultured onto an EDP-fiber network. Upon confluence by 7 days confirmed with CD31 immunostaining, as illustrated in FIGS. 3D, 3E, and 3F mouse smooth muscle cells (MOVAS) may be cultured onto the outside layer of MS-1 cells with platelet-derived growth factor (PDGFG-BB). After another 7 days' culture, the sample may be stained with CD31 antibody for endothelial cells and alpha smooth muscle actin for smooth muscle cells, as illustrated in FIG. 4D. Based on staining results, it may be seen that the MOVAS cells can wrap the fiber coated with MS-1 cells, forming a continuous supporting layer for later vessel formation.

With reference to FIGS. 6A and 6B, the exemplary microfiber templates coated with endothelial cells and mural cells may be integrated into any 3D constructs either using a layer-by-layer assembly approach (see FIG. 6B) or by being embedded in the gel (see FIG. 6A). With reference to FIG. 6A, to help direct imaging of the vascular structure, the cell-coated microfiber templates 402 may be embedded in a transparent hydrogel. That is, after the confluent endothelial layer formation 404, both the capillary culture and the arteriole/venule culture may be transferred into the 3D hydrogel matrix 406 (e.g., collagen gel) for fiber degradation and vessel lumen formation (see 408). In the embodiment shown in FIG. 6B, after laying down a thin layer of collagen gel 410 in a petri dish, microfibers encapsulated with vascular and smooth muscle cells (see 412) may be placed on top, followed by another layer of collagen gel 414. The assembly 416 may be cultured for a designated time (e.g., 1 day, 7 days, 14 days) to allow the endothelial layer to be fully integrated with the collagen construct. The microfiber may continue to degrade and may complete its degradation in no longer than 14 days. In one embodiment, the hydrogel matrix 406 and/or collagen gel 410 is also loaded with one or more types of tissue cells.

In an alternative embodiment, the method of 3D tissue integration illustrated in FIG. 6B is implemented by incorporating the microvascular network within layers of nanofiber meshes seeded with one or more types of tissue cells. In this embodiment, the 3D tissue construct 416 is made from meshes of electrospun nanofiber, and tissue cells are seeded onto these electrospun nanofiber meshes. The microvascular network mesh is then embedded between several nanofiber layers seeded with tissue cells. This method of layering nanofiber layers is more thoroughly described in U.S. patent application Ser. No. 11/985,273 (Publication No. 2008/0112998), the disclosure of which is incorporated by reference herein in its entirety.

The types of tissue cells that can be implemented using these methods include, but are not limited to, skin cells (keratinocytes, fibroblasts), bone cells (osteoblasts), heart cells (cardiomyocytes), liver cells (hepatocytes), pancreas cells (the islets of Langerhans), lung cells (alveolar epithelial cells), kidney cells (kidney epithelial cells), stomach cells (stomach epithelial cells), bladder cells (bladder epithelial cells) and intestine cells (intestinal epithelial cells).

To visualize the progression of microvascular cells in the gel, the culture may be stained with CD31 antibody and monitored under a confocal microscope. 3D images may be used to better map out the spatial distribution and the formation of a microvascular structure (e.g., lumen development) inside the collagen gel, as shown in FIGS. 2I, 2J, 3G, and 3H.

To confirm the complete degradation of the microfiber templates and the opening of the microvascular structures, fluorescently labeled dextran solution may be caused to flow through the gel constructs at the end of the culture time. Such an injection may be performed on a microinjection device, and 3D images may be taken under a confocal microscope. FIG. 7 illustrates such an image, which demonstrates that a vessel fabricated according to the exemplary embodiments may be perfusable, and that the endothelial cells' tight junctions are strong enough to perform the functions of naturally occurring blood vessels.

With reference to FIGS. 8A-8F, after the formation of confluence endothelial cells on the microfiber surface, the microfiber templates were put into 3D constructs, namely, collagen gel. A dynamic culture with a shaker was used to speed up the polymer degradation. After four weeks, samples were harvest and examined by immunostaining of laminin staining and fluorescence microscopy showed the lamina layer formed surrounding the endothelium, as seen in FIG. 8D. Hematoxylin and eosin (i.e., “H&E”) staining show the formation of empty lumen, as seen in FIG. 8E. Immunohistochemistry (IHC) followed by CD31 and laminin staining confirmed the formation of the tubes and lamina layer formed out of it, as seen in FIG. 8F.

The combination of microfiber template, endothelial cells and 3D cell assembly of the exemplary embodiments described above may allow for fabrication of a microvascular network with controlled diameters from several micrometers to tens of micrometers and even up to 100 micrometers in tissue-like constructs. Direct incorporation of angiogenic growth factors (e.g., vascular endothelial growth factor (VEGF) or basic-fibroblast growth factor (b-FGF)) into microfibers, as shown in FIGS. 9A-9D, may allow not only for customizing an endothelial cell-friendly surface with desired stimuli to facilitate lumen formation, but also for further manipulation of the microfiber degradation rate for timely release of the microvascular structure.

The exemplary embodiments may provide for cost-effective formation of various microvessel-like structures (e.g., capillaries, arterioles, venules) with sacrificial microfiber templates and different vascular cells depending on the microfiber template and the use of vascular cells. By tuning the exemplary sacrificial microfiber templates, the diameter, pattern and density of formed microvascular networks, as well as their size, can be readily controlled. By choosing different materials for the exemplary sacrificial microfibers, the time to form the exemplary vascular network can also be easily controlled. Further, exemplary microvascular networks can be readily incorporated into 3D tissue constructs by assembling the microfiber networks encapsulated with vascular cells (e.g., endothelial cells and mural cells) into the 3D constructs.

The exemplary embodiments may be useful for creation of large and complex tissues for reconstructive surgery. The exemplary embodiments may enable the creation of microvessel-like (e.g., capillaries, arterioles, venules) structures using sacrificial microfiber templates and vascular cells (e.g., endothelial cells, progenitor cells, endothelial or progenitor cells combined with mural cells such as smooth muscle cells or pericytes) with open lumens, different diameters, and arbitrary organization patterns. The exemplary embodiments may provide an approach for generating a microvascular network in 3D tissue constructs by assembling microfiber networks encapsulated with vascular cells (e.g., endothelial cells and mural cells) into the constructs by either embedding or layer-by-layer sandwiching. The microvascular networks formed through the use of the exemplary embodiments can be used to form a variety of vascularized tissues or organs such as kidney, liver, lung, muscles, periosteum, bone, fat, cardiac patches, ligament, tendon, skin, pancreas, and others.

All examples and conditional language recited herein are intended for pedagogical purposes to aid the reader in understanding the principles of the invention and the concepts contributed by the inventor to furthering the art, and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

It will be understood that the embodiments described herein are merely exemplary and that a person skilled in the art may make many variations and modifications without departing from the spirit and scope of the invention. All such variations and modifications are intended to be included within the scope of the invention, as defined by the following claims.

We claim:

1. A method of fabricating a vascular structure, comprising
providing a microfiber template, the template comprising a plurality of interconnected microfibers configured to support adhesion and spreading of endothelial cells, the plurality of interconnected microfibers being made from at least one biocompatible and biodegradable material; and
placing the microfiber template in a culture that includes at least one of endothelial cells and endothelial progenitor cells,
wherein the at least one of endothelial cells and endothelial progenitor cells seed onto the microfiber template to form at least one endothelial layer over the microfiber template, and
wherein the microfiber template at least partially degrades after a set period of time, leaving a vascular structure from the at least one endothelial layer.

2. The method of claim 1, wherein providing the microfiber template includes fabricating the microfiber template using a technique selected from the group consisting of microetching printing, electrostatic deposition prototyping, and microfluidic molding.

3. The method of claim 1, wherein the plurality of interconnected microfibers are made from at least one of solid fiber, porous fiber, and core-shell fiber.

4. The method of claim 1, wherein the at least one biocompatible and biodegradable material is selected from the group consisting of polycaprolactone, poly (L-lactic acid), poly (DL-lactic acid), poly (glycolic acid), poly(lactic-co-glycolic acid), poly(lactic-co-caprolactone), poly (dioxanone), poly (esteramide), co-poly (oxalates), poly (carbonates), poly (glutamic-co-leucine), poly (ethylene), poly(ethylene glycol)-terephthalate/poly(butylene terephthalate), poly (N-isopropylacrylamide), alginate, chitosan, collagen, gelatin, fibrinogen, elastin, silk, polysaccharide, proteoglycans, hyaluronan, laminin, and fibronectin.

5. The method of claim 1, wherein the culture further includes mural cells, and wherein the mural cells seed onto the at least one endothelial layer to form at least one smooth muscle layer over the at least one endothelial layer.

6. The method of claim 1, further comprising transferring the microfiber template with the at least one endothelial layer thereon into a three-dimensional hydrogel matrix.

7. The method of claim 6, wherein the hydrogel matrix includes at least one of collagen gel, fibrin gel, hyaluronic acid gel, alginate gel, agarose gel, chitosan gel, Matrigel matrix.

8. The method of claim 6, wherein said transferring includes placing the microfiber template with the at least one endothelial layer thereon on top of a first layer of hydrogel matrices.

9. The method of claim 8, wherein said transferring further includes placing a second layer of hydrogel matrices over the microfiber template.

10. The method of claim 1, further comprising layering the microfiber template between a first nanofiber mesh and a second nanofiber mesh, wherein the first and second nanofiber meshes are seeded with tissue cells.

11. The method of claim 10, wherein the tissue cells are selected from the group consisting of keratinocytes, fibroblasts, osteoblasts, cardiomyocytes, hepatocytes, islets of Langerhans, alveolar epithelial cells, kidney epithelial cells, stomach epithelial cells, bladder epithelial cells, and intestinal epithelial cells.

12. A template for creating vascular structures, the template comprising:
a plurality of microfibers which are interconnected to form a microfiber network, said plurality of microfibers being made from at least one biocompatible and biodegradable material, said plurality of microfibers being configured to support adhesion and spreading of endothelial cells when placed in a culture of endothelial and endothelial progenitor cells.

13. The template of claim 12, wherein said microfiber network is fabricated using a technique selected from the group consisting of microetching printing, electrostatic deposition prototyping, and microfluidic molding.

14. The template of claim 12, wherein the plurality of microfibers are made from at least one of solid fiber, porous fiber, and core-shell fiber.

15. The template of claim 12, wherein said at least one biocompatible and biodegradable material is selected from the group consisting of polycaprolactone, poly (L-lactic acid), poly (DL-lactic acid), poly (glycolic acid), poly(lactic-co-glycolic acid), poly(lactic-co-caprolactone), poly (dioxanone), poly (esteramide), co-poly (oxalates), poly (carbonates), poly (glutamic-co-leucine), poly (ethylene), poly(ethylene glycol)-terephthalate/poly(butylene terephthalate), poly (N-isopropylacrylamide), alginate, chitosan, collagen, gelatin, fibrinogen, elastin, silk, polysaccharide, proteoglycans, hyaluronan, laminin, and fibronectin.

16. The template of claim 12, wherein said plurality of microfibers are configured to support adhesion and spreading of endothelial cells and mural cells.

17. The template of claim 12, further comprising an endothelial cell layer that envelops said plurality of microfibers in said first microfiber network.

18. The template of claim 17, wherein said microfiber network is sized and shaped to occupy a volume of a capillary.

19. The template of claim 17, further comprising a smooth muscle layer attached to said endothelial cell layer.

20. The template of claim 19, wherein said microfiber network is sized and shaped to occupy a volume of an arteriole or venule.

21. The template of claim 17, further comprising a layer of hydrogel matrices seeded with tissue cells surrounding said endothelial cell layer.

22. The template of claim 21, wherein said tissue cells are selected from the group consisting of keratinocytes, fibroblasts, osteoblasts, cardiomyocytes, hepatocytes, islets of Langerhans, alveolar epithelial cells, kidney epithelial cells, stomach epithelial cells, bladder epithelial cells, and intestinal epithelial cells.

23. The template of claim 12, further comprising a first nanofiber mesh and a second nanofiber mesh, said first and second nanofiber meshes being made from electrospun nanofibers and seeded with tissue cells, wherein said microfiber network is located between said first nanofiber mesh and said second nanofiber mesh in a layered arrangement.

24. The template of claim 23, wherein said tissue cells are selected from the group consisting of keratinocytes, fibroblasts, osteoblasts, cardiomyocytes, hepatocytes, islets of Langerhans, alveolar epithelial cells, kidney epithelial cells, stomach epithelial cells, bladder epithelial cells, and intestinal epithelial cells.
